SEQUENCE LISTING (4 sequences)

SEQ ID NO: 1 - protein, 219 aa, Artificial Sequence
  Synthetic polypeptide
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, Artificial Sequence
  Synthetic polypeptide
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 113 aa, Artificial Sequence
  Synthetic polypeptide
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKR

SEQ ID NO: 4 - protein, 118 aa, Artificial Sequence
  Synthetic polypeptide
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

(19) United States
(12) Patent Application Publication
(10) Pub. No.: US 2011/0097345 A1
(43) Pub. Date: Apr. 28, 2011

(54) NOVEL DOSING REGIMEN AND METHOD OF TREATMENT

(75) Inventors: John LAMBERT, Cambridge, MA (US); James J. O'LEARY, Newton, MA (US); Joanne Elizabeth Sarah SCHINDLER, Natick, MA (US); Steven WEITMAN, Helotes, TX (US); Albert QIN, Winchester, MA (US)

(73) Assignee: IMMUNOGEN INC., Waltham, MA (US)

(21) Appl. No.: 12/909,697

(22) Filed: Oct. 21, 2010

Related U.S. Application Data
(60) Provisional application No. 61/253,804, filed on Oct. 21, 2009.

(51) Int. Cl. A61K 39/395; A61P 35/00; A61P 35/02

(52) U.S. Cl. 424/178.1

(57) ABSTRACT

This invention relates to a method of treatment and dosing regimen for treating disease, such as cancer and mammalian tumors, wherein therapy with a cytotoxic drug is suitable, by the administration of an antibody-toxin conjugate, such as a maytansinoid toxin, by infusion at an initial infusion rate of 1 mg/min or lower on a schedule selected from the group consisting of: (1) an amount of at least about 90 mg/m² on day 1 and day 8, every three weeks; (2) at least an amount of about 30 mg/m² on day 1, day 2 and day 3, every three weeks; (3) at least an amount of about 45 mg/m² on day 1, day 8, and day 15, every 4 weeks; and (4) at least an amount of about 45 mg/m² on day 1, day 8 and day 15, every 3 weeks.

### CROSS REFERENCE TO RELATED APPLICATIONS

This application claims benefit to U.S. Provisional Application Ser. No. 61/253,804, filed Oct. 21, 2009, the entire contents of which are incorporated-herein-by-reference.

### SEQUENCE LISTING

This application contains a Sequence Listing. The entire contents of the ASCII text file, file name A10420SequenceListing.txt, created Oct. 20, 2010 (file size: 8.21 KB), is incorporated-herein-by-reference.

### FIELD OF THE INVENTION

The present invention relates to a dosing regimen and method of treatment, such as, for example, a dosing method for treating a disease, such as, for example, cancer and mammalian tumors, by administration of an agent, which is a conjugate of an antibody or fragment thereof, that specifically binds to an antigen, wherein the antibody is covalently linked to a toxin, such as, for example, a maytansinoid (“antigen specific maytansinoid conjugate or immunoconjugate”). The method employs an intermittent dosing schedule to maximize, for example, the antitumor effects of the treatment with significantly higher dosages of the conjugate, while minimizing dose-limiting toxic side effects. Antibodies useful for the present invention include antibodies that specifically bind, for example, CD56, CD20, human epidermal growth factor receptor (HER1), IgE, vascular endothelial growth factor, HER dimerization inhibitors, Bc1-2 family proteins, MET, IL-13, IFN alpha, EGFL7, CD40, DR4 and DR5, PI3 kinase, lymphotoxin alpha, beta 7 integrin, amyloid beta, CRIg, TNF, complement (C5), CBL, CD147, IL-8, gp120, VLA-4, CD11a, CD18, VEGF, CD40L, Id, ICAM-1, CD2, EGFR, TGF-beta, TNF-alpha, E-selectin, Fact VII, TNF, Her2/neu, F gp, CD11/18, CD14, ICAM-3, CD80, CD40L, CD4, CD23, beta2-integrin, alpha4beta7, CD52, HLA DR, CD22, CD64 (FcR), TCR alpha beta, CD2, CD3, Hep B, CA 125, EpCAM, gp120, CMV, gpIIbIIIa, IgE, IL5, IL-4, CD25, CD3, CD33, CD30, HLA, VNRintegrin, CD25, IL-23 and IL-12. The dosing regimen involves the administration of high doses of a conjugate or conjugates by slowing the initial infusion rate of the conjugate, pre-treating patients with prophylactic agents and administering the conjugate on either of the following schedules: (1) Day 1 and Day 8 every three weeks; (2) Day 1, Day 2 and Day 3 every three weeks; or (3) Day 1, Day 8, and Day 15 every 4 weeks without substantially eliciting dose-limiting side effects such as severe head pain, and the like.

### BACKGROUND OF THE INVENTION

The treatment of various diseases has progressed significantly with the development of pharmaceuticals that more efficiently target and kill harmful cells. One intensely studied disease suitable for targeted therapy is cancer. To this end, researchers have taken advantage of cell-surface receptors and antigens selectively expressed by cancer cells to develop drugs based on antibodies that bind the tumor-specific or tumor-associated antigens. In this regard, cytotoxic molecules such as bacteria and plant toxins, radionuclides, and certain chemotherapeutic drugs have been chemically linked to antibodies that bind tumor-specific or tumor-associated cell surface antigens (see, e.g., International (PCT) Patent Application Nos. WO 00/02587, WO 02/060955, and WO 02/092127, U.S. Pat. Nos. 5,475,092, 26,340,701, 6,171,586, U.S. patent application Publication No. 2003/0004210 A1, and Ghetie et al., J. Immunol. Methods, 112, 267-277 (1988)). Such compounds are typically referred to as toxin, radionuclide, and drug “conjugates”. Often they also are referred to as immunoconjugates, radioimmunoconjugates and immunotoxins. Tumor cell killing occurs upon binding of the drug conjugate to a tumor cell and activation of the cytotoxic activity of the maytansinoid. The selectivity afforded by drug conjugates minimizes toxicity to normal cells, thereby enhancing tolerability of the drug in the patient.

In order to maximize the anti-cancer efficacy of some therapies, such as cancer treatments, it is important to maximize the dose of the anti-cancer agent in an attempt to eradicate the tumor, or at least reduce the tumor size, by killing the tumor cells in the body, while minimizing toxic dose-limiting side effects.

### SUMMARY OF THE INVENTION

The need has arisen to develop methods of administration that would overcome the limitations associated with treatment of patients with immunoconjugates, such as, for example, IMGN901, also known as lorvotuzumab mertansine, that can cause severe head pain and the like. There is a need to improve efficacy while ameliorating toxic side effects.

This invention relates to methods of treatment that address the shortcomings of the previous method of dosing and administration of conjugates. For example, the invention relates to methods of treatment that address the dosing and administration of conjugates for treating cancer and mammalian tumors.

Unexpectedly, it has been found that by slowing the initial infusion rate of the conjugate and by pre-treating patients with a prophylactic regimen of corticosteroids, the conjugate can be safely administered to patients at significantly higher doses (at least a 25% increase over an initial 6 week treatment period) and without eliciting dose-limiting severe headache, when given on a either a: (1) Day 1, 8, every 3 weeks, (2) Day 1, 2, 3, every 3 weeks or (3) Day 1, 8, 15 every 4 weeks schedule.

It is an object of the present invention to provide a method for treating cancer by maximizing the dose of the antibody conjugate, such as, for example, an antibody conjugate directed to an antigen, such as CD56, CD20, human epidermal growth factor receptor (HER1), IgE, vascular endothelial growth factor, HER dimerization inhibitors, Bc1-2 family proteins, MET, IL-13, IFN alpha, EGFL7, CD40, DR4 and DR5, PI3 kinase, lymphotoxin alpha, beta 7 integrin, amyloid beta, CRIg, TNF, complement (C5), CBL, CD147, IL-8, gp120, VLA-4, CD11a, CD18, VEGF, CD40L, Id, ICAM-1, CD2, EGFR, TGF-beta, TNF-alpha, E-selectin, Fact VII, TNF, Her2/neu, F gp, CD11/18, CD14, ICAM-3, CD80, CD40L, CD4, CD23, beta2-integrin, alpha4beta7, CD52, HLA DR, CD22, CD64 (FcR), TCR alpha beta, CD2, CD3, Hep B, CA 125, EpCAM, gp120, CMV, gpIIbIIIa, IgE, IL5, IL-4, CD25, CD3, CD33, CD30, HLA, VNRintegrin, CD25, IL-23 or IL-12, while minimizing dose-limiting toxicities.

It is another object of the present invention to provide a method for treating mammalian tumors by maximizing the antibody-maytansinoid conjugate, while minimizing dose-limiting toxicities.

It is another object of the present invention to provide a method for treating cancer such as small cell lung cancer, ovarian cancer, non small cell lung cancer, neuroendocrine tumors such as Merkel cell carcinoma, large cell neuroendocrine carcinoma of the lung, neuroendocrine tumors of the pancreas and gastro-intestinal tract; breast cancer; typical and atypical carcinoid of the lung, neuroblastoma, sarcomas including osteosarcoma, astrocytomas, Wilms tumor, schwannoma, multiple myeloma, Natural Killer (NK) cell lymphoma; acute myelocytic leukemia, any other CD56 expressing solid tumors, and any other CD56 expresing hematologic malignancies by maximizing the dose of the antibody-maytansinoid conjugate while minimizing dose-limiting toxicities.

It is another object of the present invention to provide a method for treating mammalian hematological malignancies, such as multiple myeloma, antigen positive lymphomas and leukemias, and acute myelocytic leukemia and NK cell lymphoma by maximizing the dose of the antibody-maytansinoid conjugate, while minimizing dose-limiting toxicities.

It is another object of the present invention to provide a dosing regimen for treating cancer by maximizing the dose of the antibody-maytansinoid conjugate while minimizing dose-limiting toxicities.

It is an object of the present invention to provide a dosing regimen for treating mammalian tumors by maximizing the dose of the antibody-maytansinoid conjugate while minimizing dose-limiting toxicities.

More specifically the present invention relates to a method for treating cancer with an antibody-maytansinoid conjugate, without dose-limiting toxicity, comprising pre-treating a subject in need of treatment with prophylactic corticosteroids and subsequently administering the antibody-maytansinoid conjugate by infusion at an initial infusion rate of 1 mg/min or lower on a schedule selected from the group consisting of: (1) an amount of at least about 90 mg/m² on day 1 and day 8, every three weeks; (2) an amount of at least about 30 mg/m² on day 1, day 2 and day 3, every three weeks; and (3) an amount of at least about 60 mg/m² on day 1, day 8, and day 15, every 4 weeks.

In another aspect, the present invention relates to a method for treating mammalian tumors without dose-limiting toxicity, comprising pre-treating a subject in need of treatment with prophylactic corticosteroids and subsequently administering an antibody-maytansinoid conjugate, by infusion at an initial infusion rate of 1 mg/min or lower on a schedule selected from the group consisting of: (1) an amount of at least about 90 mg/m² on day 1 and day 8, every three weeks; (2) an amount of at least about 30 mg/m² on day 1, day 2 and day 3, every three weeks; and (3) an amount of at least about 60 mg/m² on day 1, day 8, and day 15, every 4 weeks.

The present invention also relates to a dosing regimen for use in the treatment of cancer and mammalian tumors by administration of an antibody-maytansinoid conjugate, to maximize the dose of the anti-cancer agent, while minimizing dose-limiting toxicities.

More specifically the present invention relates to a dosing regimen for the treatment of cancer without dose-limiting toxicity, comprising pre-treating a subject in need of treatment with prophylactic corticosteroids and subsequently administering an antibody-maytansinoid conjugate, by infusion at an initial infusion rate of 1 mg/min or lower on a schedule selected from the group consisting of: (1) an amount of at least about 90 mg/m² on day 1 and day 8, every three weeks; (2) an amount of at least about 30 mg/m² on day 1, day 2 and day 3, every three weeks; and (3) an amount of at least about 60 mg/m² on day 1, day 8, and day 15, every 4 weeks.

In another aspect, the present invention relates to a dosing regimen for treating mammalian tumors without dose-limiting toxicity, comprising pre-treating a subject in need of treatment with prophylactic corticosteroids and subsequently administering an antibody-maytansinoid conjugate, by infusion at an initial infusion rate of 1 mg/min on a schedule selected from the group consisting of: (1) an amount of at least about 90 mg/m² on day 1 and day 8, every three weeks; (2) an amount of at least about 60 mg/m² on day 1, day 2 and day 3, every three weeks; and (3) an amount of at least about 60 mg/m² on day 1, day 8, and day 15, every 4 weeks.

In yet another aspect, the present invention relates to a dosing regimen for treating mammalian tumors such as antigen positive hematologic malignancies, without dose-limiting toxicity, comprising pre-treating a subject in need of treatment with prophylactic corticosteroids and subsequently administering an antibody-maytansinoid conjugate, in combination with a second anti-cancer agent by infusion at an initial infusion rate of 1 mg/min or lower on a schedule of (1) at least an amount of about 45 mg/m² on day 1, day 8, and day 15, every 4 weeks or (2) at least an amount of about 45 mg/m² on day 1 and day 8, every three weeks.

In the present invention, if tolerated, the initial infusion rate of 1 mg/min or lower can be increased up to 3 mg/min, preferably incremental increases, and more preferably in increments of 0.5 mg/min. The initial infusion rate is tolerated if, after administration of the initial dose of 1 mg/min or lower for 15 minutes, the subject shows signs or symptoms of not more than moderate intensity (or <grade 2 NCI CTCAE criteria) (See, Common Terminology Criteria for Adverse Events, Version 4.0, May 28, 2009, Version 4.03, Jun. 14, 2010; U.S. Dept. Health and Human Services), which is incorporated herein by reference in its entirety).

The dosing regimens of the invention can yield dose intensities of the antibody-maytansinoid of at least about 360 mg/m² over 6 weeks and at least about 540 mg/m² over 12 weeks.

In preferred aspects, the antigen is CD56 and the anti-CD56-maytansinoid conjugate is IMGN901.

A further aspect is a method wherein CD20 is the antigen.

A further aspect is a method wherein human epidermal growth factor receptor is the antigen.

A further aspect is a method wherein IgE is the antigen.

A further aspect is a method wherein vascular endothelial growth factor is the antigen.

A further aspect is a method wherein a HER dimerization inhibitor is the antigen.

A further aspect is a method wherein a Bc1-2 family protein is the antigen.

A further aspect is a method wherein any one of MET, IL-13, IFN alpha, EGFL7, CD40, DR4 and DR5, PI3 kinase, lymphotoxin alpha, beta 7 integrin, amyloid beta, CRIg, TNF, complement (C5), CBL, CD147, IL-8, gp120, VLA-4, CD11a, CD18, VEGF, CD40L, Id, ICAM-1, CD2, EGFR, TGF-beta, TNF-alpha, E-selectin, Fact VII, TNF, Her2/neu, F gp, CD11/18, CD14, ICAM-3, CD80, CD40L, CD4, CD23, beta2-integrin, alpha4beta7, CD52, HLA DR, CD22, CD64 (FcR), TCR alpha beta, CD2, CD3, Hep B, CA 125, EpCAM, gp120, CMV, gpIIbIIIa, IgE, IL5, IL-4, CD25, CD3, CD33, CD30, HLA, VNR integrin, CD25, IL-23 or IL-12 is the antigen.

### DETAILED DESCRIPTION OF THE INVENTION

The conjugate of the present invention is an immunoconjugate synthesized by the conjugation of the cytotoxic drug, such as a maytansinoid, to an antibody or an antigen binding fragment thereof.

As used herein, an “antibody” and the like also includes any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to an antigen, for example, such as CD56. For example, antibody fragments capable of binding to CD56, including, but not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology).

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein. Resurfacing or CDR grafting of antibodies can be performed as disclosed in U.S. Pat. No. 5,639,641, which is entirely incorporated herein by reference.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Antibodies useful for the present invention include antibodies that specifically bind, for example, CD56, CD20, human epidermal growth factor receptor (HER1), IgE, vascular endothelial growth factor, HER dimerization inhibitors, Bc1-2 family proteins, MET, IL-13, IFN alpha, EGFL7, CD40, DR4 and DR5, PI3 kinase, lymphotoxin alpha, beta 7 integrin, amyloid beta, CRIg, TNF, complement (C5), CBL, CD147, IL-8, gp120, VLA-4, CD11a, CD18, VEGF, CD40L, Id, ICAM-1, CD2, EGFR, TGF-beta, TNF-alpha, E-selectin, Fact VII, TNF, Her2/neu, F gp, CD11/18, CD14, ICAM-3, CD80, CD40L, CD4, CD23, beta2-integrin, alpha4beta7, CD52, HLA DR, CD22, CD64 (FcR), TCR alpha beta, CD2, CD3, Hep B, CA 125, EpCAM, gp120, CMV, gpIIbIIIa, IgE, IL5, IL-4, CD25, CD3, CD33, CD30, HLA, VNRintegrin, CD25, IL-23 and IL-12.

The antibody or fragment thereof is preferably a human, resurfaced chimeric or humanized antibody. More specifically, the antibody can be a resurfaced or humanized murine N901 antibody or fragment thereof, wherein the N901 antibody comprises a heavy chain and a light chain, said heavy chain comprising three complementarity determining regions comprising HCCDR1, HCCDR2 and HCCDR3 of murine antibody N901, and said light chain comprising three complementarity determining regions comprising LCCDR1, LCCDR2 and LCCDR3 of murine antibody N901. Even more specifically, the resurfaced antibody is huN901 or an antigen binding fragment thereof. Even more specifically, the amino acid sequence of huN901 is known in the art. Below, for example, are shown the amino acid sequences of the full length huN901 light and heavy chains and the amino acid sequences of the light and heavy chain variable regions however, amino acid sequences useful in the present invention are known in the art, such as, for example, Roguska et al. (Proc. Natl. Acad. Sci. USA, Vol. 91, pp 969-973, February 1994), U.S. Pat. No. 7,342,110 and U.S. Pat. No. 5,552,293, the contents of which are incorporated herein by reference in their entirety.

huN901 Light
(SEQ ID NO: 1)
DVVMTQSPLSLPVTLGQPASISCRSSQIIIHSDGNTYLEWFQQRPGQSPR
RLIYKVSNRFSGVPDRFSGSGSGTDFTLKISRVEAEDVGVYYCFQGSHVP
HTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.
huN901 Heavy
(SEQ ID NO: 2)
QVQLVESGGGVVQPGRSLRLSCAASGFTFSSFGMHWVRQAPGKGLEWVAY
ISSGSFTIYYADSVKGRFTISRDNSKNTLYLQMNSLRAEDTAVYYCARMR
KGYAMDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK.
huN901LCV
(SEQ ID NO: 3)
DVVMTQSPLSLPVTLGQPASISCRSSQIIIHSDGNTYLEWFQQRPGQSPR
RLIYKVSNRFSGVPDRFSGSGSGTDFTLKISRVEAEDVGVYYCFQGSHVP
HTFGQGTKVEIKR.
huN901HCV
(SEQ ID NO: 4)
QVQLVESGGGVVQPGRSLRLSCAASGFTFSSFGMHWVRQAPGKGLEWVAY
ISSGSFTIYYADSVKGRFTISRDNSKNTLYLQMNSLRAEDTAVYYCARMR
KGYAMDYWGQGTLVTVSS.

In one aspect of the invention, the above-described antibody is chemically coupled to a cytotoxic drug, such as a maytansinoid.

In one aspect of the invention the toxin conjugate is a maytansinoid. Maytansinoids were originally isolated from the east African shrub belonging to the genus Maytenus, but were subsequently also discovered to be metabolites of soil bacteria, such as Actinosynnema pretiosum (see, e.g., U.S. Pat. No. 3,896,111). Maytansinoids induce cytotoxicity through mitotic inhibition. Experimental evidence suggests that maytansinoids inhibit mitosis by inhibiting polymerization of the microtubule protein tubulin, thereby preventing formation of microtubules (see, e.g., U.S. Pat. No. 6,441,163 and Remillard et al., Science, 189, 1002-1005 (1975)). Maytansinoids have been shown to inhibit tumor cell growth in vitro using cell culture models, and in vivo using laboratory animal systems. Moreover, the cytotoxicity of maytansinoids is 1,000-fold greater than conventional chemotherapeutic agents, such as, for example, methotrexate, daunorubicin, and vincristine (see, e.g., U.S. Pat. No. 5,208,020). Maytansinoids are known in the art to include maytansine, maytansinol, C-3 esters of maytansinol, and other maytansinol analogues and derivatives (see, e.g., U.S. Pat. Nos. 5,208,020 and 6,441,163). C-3 esters of maytansinol can be naturally occurring or synthetically derived. Moreover, both naturally occurring and synthetic C-3 maytansinol esters can be classified as a C-3 ester with simple carboxylic acids, or a C-3 ester with derivatives of N-methyl-L-alanine, the latter being more cytotoxic than the former. Synthetic maytansinoid analogues also are known in the art and described in, for example, Kupchan et al., J. Med. Chem., 21, 31-37 (1978). Methods for generating maytansinol and analogues and derivatives thereof are described in, for example, U.S. Pat. No. 4,151,042.

Suitable maytansinoids for use in the invention can be isolated from natural sources, synthetically produced, or semi-synthetically produced using methods known in the art. Moreover, the maytansinoid can be modified in any suitable manner, so long as sufficient cytotoxicity is preserved in the ultimate conjugate molecule. In this regard, maytansinoids lack suitable functional groups to which antibodies can be linked.

A linking moiety is utilized to link the maytansinoid to the antibody to form the conjugate. The linking moiety contains a chemical bond that allows for the activation of maytansinoid cytotoxicity at a particular site. Suitable chemical bonds are well known in the art and include disulfide bonds, acid labile bonds, photolabile bonds, peptidase labile bonds, thioether bonds formed between sulfhydryl and maleimide groups, and esterase labile bonds. Most preferably, the linking moiety comprises a disulfide bond. In accordance with the invention, the linking moiety preferably comprises a reactive chemical group. Particularly preferred reactive chemical groups are N-succinimidyl esters and N-sulfosuccinimidyl esters. In a preferred aspect, the reactive chemical group can be covalently bound to the maytansinoid via disulfide bonding between thiol groups. Thus, a maytansinoid modified as described herein preferably comprises a thiol group. One of ordinary skill in the art will appreciate that a thiol group contains a sulfur atom bonded to a hydrogen atom and is typically also referred to in the art as a sulfhydryl group, which can be denoted as “—SH” or “RSH.”

Particularly preferred are maytansinoids comprising a linking moiety that contains a reactive chemical group are C-3 esters of maytansinol and its analogs where the linking moiety contains a disulfide bond and the chemical reactive group comprises a N-succinimidyl or N-sulfosuccinimidyl ester. Many positions on maytansinoids can serve as the position to chemically link the linking moiety. For example, the C-3 position having a hydroxyl group, the C-14 position modified with hydroxymethyl, the C-15 position modified with hydroxy and the C-20 position having a hydroxy group are all useful. The linking moiety most preferably is linked to the C-3 position of maytansinol. Most preferably, the maytansinoid used in connection with the invention is N^{2′}-deacetyl-N^{2′}-(3-mercapto-1-oxopropyl)-maytansine (DM1), N^{2′}-deacetyl-N-^{2′}(4-mercapto-1-oxopentyl)-maytansine (DM3), or N^{2′}-deacetyl-N^{2′}-(4-mercapto-4-methyl-1-oxopentyl)-maytansine (DM4).

Linking moieties with other chemical bonds also can be used in the context of the invention, as can other maytansinoids. Specific examples of other chemical bonds include acid labile bonds, thioether bonds, photolabile bonds, peptidase labile bonds and esterase labile bonds. Methods for producing maytansinoids with linking moieties are described in, for example, U.S. Pat. Nos. 5,208,020, 5,416,064, and 6,333,410.

Cleavable linkers are linkers that can be cleaved under mild conditions, i.e. conditions under which the activity of the maytansinoid drug is not affected. Many known linkers fall in this category and are described below.

Disulfide containing linkers are linkers cleavable through disulfide exchange, which can occur under physiological conditions.

Acid-labile linkers are linkers cleavable at acid pH. For example, certain intracellular compartments, such as endosomes and lysosomes, have an acidic pH (pH 4-5), and provide conditions suitable to cleave acid-labile linkers.

Linkers that are photo-labile are useful at the body surface and in many body cavities that are accessible to light. Furthermore, infrared light can penetrate tissue.

Some linkers can be cleaved by peptidases. Only certain peptides are readily cleaved inside or outside cells, see e.g. Trouet et al., 79 Proc. Natl. Acad. Sci. USA, 626-629 (1982) and Umemoto et al. 43 Int. J. Cancer, 677-684 (1989). Furthermore, peptides are composed of α-amino acids and peptidic bonds, which chemically are amide bonds between the carboxylate of one amino acid and the α-amino group of a second amino acid. Other amide bonds, such as the bond between a carboxylate and the ε-amino group of lysine, are understood not to be peptidic bonds and are considered non-cleavable.

Some linkers can be cleaved by esterases. Again only certain esters can be cleaved by esterases present inside or outside cells. Esters are formed by the condensation of a carboxylic acid and an alcohol. Simple esters are esters produced with simple alcohols, such as aliphatic alcohols, and small cyclic and small aromatic alcohols. For example, the present inventors found no esterase that cleaved the ester at C-3 of maytansine, since the alcohol component of the ester, maytansinol, is very large and complex.

A non-cleavable linker is any chemical moiety that is capable of linking a maytansinoid to a cell-binding agent in a stable, covalent manner and does not fall under the categories listed above as cleavable linkers. Thus, non-cleavable linkers are substantially resistant to acid-induced cleavage, light-induced cleavage, peptidase-induced cleavage, esterase-induced cleavage, and disulfide bond cleavage.

“Substantially resistant” to cleavage means that the chemical bond in the linker or adjoining the linker in at least 80%, preferably at least 85%, more preferably at least 90%, even more preferably at least 95%, and most preferably at least 99% of the cell-binding agent maytansinoid conjugate population remains non-cleavable by an acid, a photolabile-cleaving agent, a peptidase, an esterase, or a chemical or a physiological compound that cleaves the chemical bond (such as a disulfide bond) in a cleavable linker, for within a few hours to several days of treatment with any of the agents described above.

Furthermore, “non-cleavable” refers to the ability of the chemical bond in the linker or adjoining to the linker to withstand cleavage induced by an acid, a photolabile-cleaving agent, a peptidase, an esterase, or a chemical or a physiological compound that cleaves a disulfide bond, at conditions under which the maytansinoid or the cell binding agent does not lose its activity.

A person of ordinary skill in the art would readily distinguish non-cleavable from cleavable linkers.

Particularly preferred linker molecules include, for example, N-succinimidyl 3-(2-pyridyldithio)propionate (SPDP) (see, e.g., Carlsson et al., Biochem. J., 173, 723-737 (1978)), N-succinimidyl 4-(2-pyridyldithio)butanoate (SPDB) (see, e.g., U.S. Pat. No. 4,563,304), N-succinimidyl 4-(2-pyridyldithio)pentanoate (SPP) (see, e.g., CAS Registry number 341498-08-6), N-succinimidyl 4-(N-maleimidomethyl)cyclohexane-1-carboxylate (SMCC) (see, e.g., Yoshitake et al., Eur. J. Biochem., 101, 395-399 (1979)), and N-succinimidyl 4-methyl-4-[2-(5-nitro-pyridyl)-dithio]pentanoate (SMNP) (see, e.g., U.S. Pat. No. 4,563,304). The most preferred linker molecules for use in the invention are SPP and SPDB.

One aspect of the invention is IMGN901 (huN90′-DM1), an immunoconjugate synthesized by the conjugation of the cytotoxic maytansinoid drug DM1 to a resurfaced version of the murine monoclonal antibody N901. On average, there are about 3.5 molecules of DM1 linked to each antibody molecule. Methods for the preparation and formulation of IMGN901 have been described in U.S. Pat. No. 7,374,762, U.S. Pub. App. No. 2007/0031402, U.S. Pub. App. no. 2007/0048314 and U.S. Pub. App. No. 2006/0182750, each of which is incorporated by reference herein in its entirety.

IMGN901 (huN90′-DM1) is an exemplary aspect of the invention. In preclinical studies, IMGN901 shows 100-1000 fold higher potency than conventional cytotoxics. Another characteristic feature of IMGN901 is the attachment of the maytansinoid to the antibody by disulfide linkage, providing a conjugate that is stable in blood plasma, yet readily cleaved within the target cells to which the antibody binds.

The nomenclature and structure of IMGN901 are shown below.

Code Name: IMGN901

Common Name: Maytansinoid DM1-conjugated humanized monoclonal antibody huN901

Other Names: huN901-DM1, IMGN901, BB-10901, lorvotuzumab mertansine

Chemical Name: N^{2′}-deacetyl-N^{2′}-(3-mercapto-1-oxopropyl)-maytansin conjugated humanized monoclonal antibody N901.

A schematic representation of DM1 bound to the humanized N901 antibody is shown below, wherein n, on average, equals about 3.5.

IMGN901 (Maytansinoid DM1 conjugated to huN901 antibody)

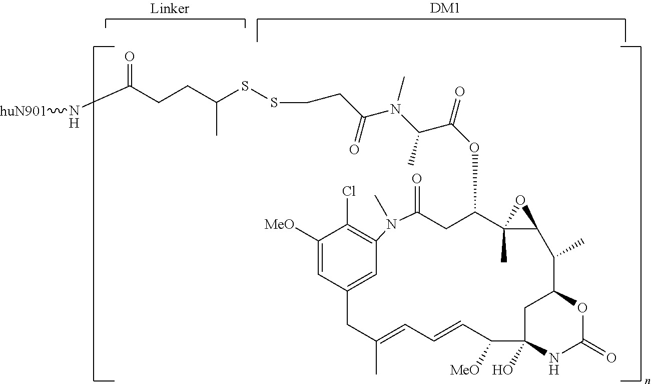

IMGN901 binds with high affinity to CD56, an antigen of the family of neural cell adhesion molecules (NCAMs) (Aletsee-Ufrecht et al., 1990 FEBS Lett 267:295). Once bound to CD56, the conjugate is internalized and releases DM1. Released DM1 inhibits tubulin polymerization and microtubule assembly, causing cell death. IMGN901 is referred to as a Tumor-Activated Prodrug (TAP) since the conjugation of DM1 to huN901 renders the cytotoxic drug inactive until it reaches the target site.

CD56 is an exemplary antigen of the invention. CD56 is expressed on a variety of tumor types including solid tumors such as small cell lung carcinoma and neuroendocrine tumors as well as hematological malignancies such as multiple myeloma (about 70% of subjects) and acute myelocytic leukemia (Aletsee-Ufrecht et al., 1990 FEBS Lett 267:295). Among subjects with multiple myeloma (MM), gene expression profiles of primary multiple myeloma cells demonstrated that CD56 is expressed in 10 of 15 subjects (66.6%) and flow cytometric profiles of MM cells revealed CD56 expression in 22 of 28 subjects (Tassone et al., Cancer Res 2004 64:4629).

The expression profile of CD56 for hematological cells is restricted to Natural Killer (NK) cells and a subset of T lymphocytes that express the NCAM glycoprotein. CD56 is expressed in malignant plasma cells, but it is not expressed on normal plasma cells. The restricted expression of CD56 in the normal hematopoietic compartment combined with its expression on malignant plasma cells provides a conceptual basis for evaluating CD56 as a target for immunoconjugate based therapy in multiple myeloma.

Findings from non-clinical studies reveal that IMGN901 has highly significant anti-tumor activity at doses that are well tolerated in mouse xenograft tumor models of small cell lung cancer, ovarian cancer, non small cell lung cancer, neuroendocrine tumors such as merkel cell carcinoma, typical and atypical carcinoid of the lung large cell neuroendocrine carcinoma of the lung, breast cancer, neuroblastoma, osteosarcoma and other sarcomas, astrocytomas, wilms tumor, and schwannoma.

In a Phase I clinical study of IMGN901 in patients with CD56+ tumors, the maximally tolerable dose of IMGN901 was 60 mg/m²/week×4 doses every six weeks, for a maximal dose intensity of 240 mg/m² every 6 weeks (Tolcher et al., November 2002, 14th EORTC-NCI-AACR Symposium on Molecular Targets and Cancer Therapeutics “A Phase I and Pharmacokinetic Study of BB10901, a maytansinoid immunoconjugate, in CD56 expressing tumors”). The principal dose-limiting toxicity was severe aseptic meningitis-like headache, observed in 2 of 4 patients treated at 75 mg/m²/week and 1 of 4 patients treated at 67.5 mg/m²/week. Although transient in nature (<24 hr duration), the headaches were severe (Grade 3, debilitating) and prevented further dosing with IMGN901.

The biological basis for the dose-limiting toxicity of IMGN901 may relate to an impact of IMGN901 treatment on CD56-expressing cells in the nervous system and/or hematopoietic cells. Several other antibody-DM1 immunoconjugates which target other antigens (not CD56) did not elicit severe head pain, such as severe aseptic meningitis-like headaches and the like in Phase I studies, even at higher doses (Tolcher et al., J Clin Oncol 2003 21:211; Galsky et al., J Clin Oncol 2008 26:2147).

Severe headache and the like, the dose-limiting toxicity of IMGN901, is clearly distinct from well-described infusion related toxicities associated with many monoclonal antibody therapies, including rituximab, trastuzumab, and cetuximab. Administration of these approved antibody therapies are associated with cytokine infusion reactions or hypersensitivity reactions, which vary in their severity and intensity across patients (Chung, The Oncologist 2008, 13:725). Infusion reactions are characterized by fever, chills, flushing, and nausea, with the onset of symptoms occurring during the infusion or immediately thereafter. Premedication with antihistamines and corticosteroids has been reported to reduce the incidence or severity of antibody-mediated infusion related toxicities. For example, patients pretreated with antihistamine plus corticosteroid had fewer, and less severe infusion reactions to cetuximab (Siena et al., J Clin Oncol 2007; 25(18 suppl); Abstract 4137). Generally, such prophylactic regimens have been used with antibody therapeutics to manage infusion-related toxicities that are not dose-limiting. It would therefore not have been expected that similar prophylactic regimens could prevent severe head pain, a distinct toxicity defined as the dose-limiting toxicity of IMGN901, or enable a substantially higher IMGN901 dose intensity in patients.

Prior to the present invention, the maximally tolerable dose of IMGN901 was reported to be 60 mg/m² administered by infusion at an initial infusion rate of 3 mg/min on days 1, 8, 15 and 22 every six weeks. The maximal dose intensity was about 240 mg/m² over 6 weeks (Tolcher et al., EORTC, November 2002). The dose intensity for 2 treatment cycles was 480 mg/m² over 12 weeks.

In order to maximize the anti-cancer efficacy of cancer treatments, it is important to maximize the dose of the anti-cancer agent in an attempt to eradicate the tumor, or at least to reduce the tumor size, by killing tumor cells in the body while minimizing toxic side effects (Le Tourneau et al., JNCI-2009 101:708). Unexpectedly, it was found that by slowing the initial infusion rate of, for example, IMGN901 and by pre-treating patients with a prophylactic regimen of corticosteroids or corticosteroids in combination with antihistamines, IMGN901, for example, can be safely administered to patients at significantly higher doses (at least a 25% increase over an initial 6 week treatment period) and without eliciting dose-limiting severe headache, when given on either a (1) Day 1, 8, every 3 weeks, (2) Day 1, 2, 3, every 3 weeks or (3) Day 1, 8, 15 every 4 weeks schedule. Dosing schedules (1) and (2) can yield dose intensities of at least about 360 mg/m², over 6 weeks and dosing schedule (3) can yield a dose intensity of at least about 540 mg/m² over 12 weeks.

Known compositions comprising a therapeutically effective amount of the antibody-maytansinoid conjugate may be used in the present invention. A “therapeutically effective amount” means an amount sufficient to show a meaningful benefit in an individual, e.g., promoting at least one aspect of tumor cell cytotoxicity, or treatment, healing, prevention, or amelioration of other relevant medical condition(s) associated with a particular cancer. Therapeutically effective amounts may vary depending upon the biological effect desired in the individual, condition to be treated, and/or the specific characteristics of the conjugate, and the individual. Thus, in accordance with the methods described herein, the attending physician (or other medical professional responsible for administering the composition) will typically decide the amount of the composition with which to treat each individual patient.

The antibody-maytansinoid conjugate is desirably formulated into a composition acceptable for pharmaceutical use, such as, for example, administration to a human host in need thereof. To this end, the conjugate molecule preferably is formulated into a composition comprising a physiologically acceptable carrier (e.g., excipient or diluent). Physiologically acceptable carriers are well known and are readily available, and include buffering agents, anti-oxidants, bacteriostats, salts, and solutes that render the formulation isotonic with the blood or other bodily fluid of the human patient, and aqueous and non-aqueous sterile suspensions that can include suspending agents, solubilizers, thickening agents, stabilizers (e.g., surfactants), and preservatives. The choice of carrier will be determined, at least in part, by the location of the target tissue and/or cells, and the particular method used to administer the composition. Examples of suitable carriers and excipients for use in drug conjugate formulations are disclosed in, for example, International (PCT) Patent Application Nos. WO 00/02587, WO 02/060955, and WO 02/092127, and Ghetie et al., J. Immunol. Methods, 112, 267-277 (1988). Most preferably, the composition comprises a buffering agent, a surfactant, a tonicifying amount of sodium chloride, and water.

In an aspect of the invention, the composition comprises (i) about 5 mg/mL of a conjugate comprising huN901 chemically coupled to DM1, (ii) about 10 mM sodium citrate buffer, (iii) about 0.01% polysorbate 20, (iv) about 120 mM sodium chloride, and (v) water (preferably water suitable for injection (WFI)), wherein the pH of the composition is about 5.5.

Compositions containing antibodies (or proteins in general) are rendered unstable by oxidation. Thus, in another aspect of the invention, the composition further comprises an antioxidant. Any suitable antioxidant can be used in the composition. Suitable antioxidants are known in the art and include, for example, superoxide dismutase, glutathione peroxidase, tocotrienols, polyphenols, zinc, manganese, selenium, vitamin C, vitamin E, beta carotene, cysteine, and methionine. The antioxidant used in connection with the composition most preferably is methionine. The antioxidant can be present in the composition in any suitable concentration.

In addition to antioxidants, the composition can further be stabilized by the addition of sucrose. The use of sucrose to stabilize antibody formulations is known to those of skill in the art. Any suitable amount of sucrose can be used in the composition.

In addition to the water-containing composition described herein (also referred to herein as a “liquid” or “aqueous” composition), the conjugate can be contained in a lyophilized composition comprising (i) a therapeutically effective amount of a conjugate comprising an antibody chemically coupled to a maytansinoid, (ii) a buffering agent, (iii) a surfactant, (iv) a cryoprotectant, and (v) a bulking agent, wherein the composition has a pH of about 5-6 when reconstituted with water. By “lyophilized” is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying). Descriptions of the conjugate (i.e., the antibody chemically coupled to the maytansinoid), buffering agent, surfactant, and components thereof, set forth above in connection with other aspects of the invention also are applicable to those same aspects of the aforesaid lyophilized composition. Prior to reconstitution of the lyophilized composition, the relative amounts of each component comprising the lyophilized composition can be described in terms of mg of excipient (e.g., buffer, surfactant, bulking agent, cryoprotectant) per mg of conjugate.

In order to prevent degradation of the active ingredients of the composition during freezing and drying, the lyophilized composition further comprises a cryoprotectant, preferably an amorphous cryoprotectant. The term “cryoprotectant,” as used herein, refers to an excipient that protects unstable molecules during freezing. Suitable cryoprotectants for use in the composition are known to those skilled in the art, and include, for example, glycerol, dimethyl sulfoxide (DMSO), polyethylene glycol (PEG), dextran, glucose, trehalose, and sucrose. Most preferably, the cryoprotectant is sucrose. The cryoprotectant may be present in the lyophilized composition in any suitable amount.

The lyophilized composition can further contain a bulking agent, preferably a crystallizable bulking agent. Bulking agents typically are used in the art to provide structure and weight to the “cake” produced as a result of lyophilization. Any suitable bulking agent known in the art may be used in connection with the lyophilized composition. Suitable bulking agents include, for example, mannitol, dextran, and glycine. The bulking agent used in the composition most preferably is glycine. The lyophilized composition can contain any suitable amount of the bulking agent.

Thus, in accordance with the invention, the contents of a lyophilized composition that is to be reconstituted to contain 5 mg/mL of conjugate (e.g., preferably a conjugate comprising an antibody, such as, for example, huN901 chemically coupled to DM1) preferably comprises (i) about 0.3 mg sodium succinate buffer per mg of the conjugate, (ii) about 0.02 mg polysorbate 20 per mg of the conjugate, (iii) about 1 mg sucrose per mg of the conjugate, and (iv) about 3.8 mg glycine per mg of the conjugate. Once reconstituted with water, such a lyophilized composition preferably has a pH of about 5.5. Moreover, when the lyophilized composition is reconstituted with water, the descriptions of the relative concentrations of the conjugate, the buffering agent, and the surfactant set forth above in connection with the liquid composition also are applicable to the aforesaid lyophilized composition.

In addition to the preferred aspects described herein, the composition (whether in liquid or lyophilized form) can comprise additional therapeutic or biologically active agents. For example, therapeutic factors useful in the treatment of a particular indication (e.g., cancer) can be present. Factors that control inflammation, such as ibuprofen or corticosteroids, can be part of the composition to reduce swelling and inflammation associated with in vivo administration of the composition and physiological distress. Immune enhancers can be included in the composition to up regulate the body's natural defenses against disease. Vitamins and minerals, antioxidants, and micronutrients can be co-administered with the composition. Antibiotics, i.e., microbicides and fungicides, can be present to reduce the risk of infection pertaining to the procedures associated with administration of the composition and other disorders.

The inventive method involves administering the conjugate to a human.

While any suitable means of administering the composition to a human can be used within the context of the invention, typically and preferably the composition is administered to a human via injection, and most preferably via infusion. By the term “injection,” it is meant that the composition is forcefully introduced into a target tissue of the human. By the term “infusion,” it is meant that the composition is introduced into a tissue, typically and preferably a vein, of the human. The composition can be administered to the human by any suitable route, but preferably is administered to the human intravenously or intraperitoneally. When the inventive method is employed to kill tumor cells, however, intratumoral administration is particularly preferred. When the composition is administered by injecting, any suitable injection device can be used to administer the composition directly to a tumor. For example, the common medical syringe can be used to directly inject the composition into a subcutaneous tumor.

In a first aspect of the invention the antibody-maytansinoid conjugate, such as, for example, IMGN901, is administered on a schedule of Day 1 and Day 8, every 3 weeks.

In the first aspect, the exemplary anti-CD56-maytansinoid conjugate, such as IMGN901 is administered intravenously as single-agent therapy on days 1 and 8 every 21 days at a dose of at least about 90 mg/m². The exemplary anti-CD56-maytansinoid conjugate is infused initially at the rate of 1 mg/min or lower. If tolerated, the infusion rate may be subsequently increased up to 3 mg/min, preferably in increments and more preferably in increments of 0.5 mg/min. Preferable doses of the conjugate are 90 mg/m² and 112 mg/m², in a given course. Treatment with the exemplary anti-CD56-maytansinoid conjugate, such as IMGN901, is preceded by a prophylactic regimen of corticosteroids on the day prior to administration of the anti-CD56-maytansinoid conjugate and on the day of administration prior to the infusion, preferably about one hour prior to the infusion, patients should also receive premedication with corticosteroids. This dosing schedule can yield a dose intensity of the anti-CD56-maytansinoid conjugate of at least about 360 mg/m² over 6 weeks.

In a second aspect of the invention the exemplary anti-CD56-maytansinoid conjugate, such as IMGN901, is administered on a schedule of day 1, day 2, and day 3, every 3 weeks.

In the second aspect, the exemplary anti-CD56-maytansinoid conjugate is administered intravenously as single-agent therapy daily for 3 days every 3 weeks. The anti-CD56-maytansinoid conjugate, such as IMGN901, is administered at a dose of at least about 30 mg/m². The anti-CD56-maytansinoid conjugate is infused initially at the rate of 1 mg/min or lower. If tolerated, the infusion rate may be subsequently increased up to 3 mg/min, preferably in increments and more preferably in increments of 0.5 mg/min. Preferred doses of the conjugate are 30 mg/m², 36 mg/m², 48 mg/m², 60 mg/m², and 75 mg/m² and 94 mg/m², in a given course. Treatment with the anti-CD56-maytansinoid conjugate should be preceded by a prophylactic regimen of corticosteroids on the day prior to administration of the anti-CD56-maytansinoid conjugate and on the day of administration, preferably about one hour prior to the infusion. This dosing schedule can yield a dose intensity of the anti-CD56-maytansinoid conjugate of at least about 360 mg/m² over 6 weeks.

In a third aspect of the invention, the exemplary anti-CD56-maytansinoid conjugate, such as IMGN901, treatment is administered on a schedule of day 1, day 8, and day 15, every 4 weeks.

The exemplary anti-CD56-maytansinoid conjugate is administered intravenously as a single agent on days 1, 8, and 15 every 4 weeks. The exemplary anti-CD56-maytansinoid conjugate is administered at a dose of at least about 60 mg/m². The exemplary anti-CD56-maytansinoid conjugate is infused initially at the rate of 1 mg/min or lower. If tolerated, the infusion rate may be subsequently increased up to 3 mg/min, preferably in increments and more preferably in increments of 0.5 mg/min. Preferred doses of the conjugate are 60 mg/m², 75 mg/m², 90 mg/m² and 112 mg/m², in a given course. Treatment with the anti-CD56-maytansinoid conjugate should be preceded by a prophylactic corticosteroid regimen on the day prior to and on the day of administration, preferably about one hour prior to infusion of the anti-CD56-maytansinoid conjugate. This dosing schedule can yield a dose intensity of the anti-CD56-maytansinoid conjugate at least about 540 mg/m² over 12 weeks.

In a fourth aspect of the invention the conjugate, such as IMGN901, treatment is administered on a schedule of day 1, day 8, and day 15, every 4 weeks or on day 1 and day 8 every three weeks in combination with other anti-cancer agents or other anticancer treatment. Anti-cancer agent means one or more agents used in the treatment of cancer alone or in combination. Similarly, anticancer treatment means one or more treatments, regimens, or therapies used in the treatment of cancer, alone or in combination. For example, exemplary IMGN901 in combination with lenalidomide and dexamethasone or in combination with etoposide and carboplatin are indicated for the treatment of CD56 positive hematologic malignancies.

In the fourth aspect, the conjugate, such as IMGN901, is administered intravenously in combination with another anti-cancer agent, such as lenalidomide and dexamethasone or etoposide and carboplatin. IMGN901 is administered on days 1, 8, and 15, every 4 weeks at a dose of at least about 45 m g/m², or at least an amount of about 45 mg/m² on day 1 and day 8, every three weeks. The conjugate is infused initially at a rate of 1 mg/min or lower. If tolerated, the infusion rate may be subsequently increased up to 3 mg/min, preferably in increments and more preferably in increments of 0.5 mg/min. Preferred doses of IMGN901 are 60 mg/m², 75 mg/m², 90 mg/m² and 112 mg/m², in a given course. Treatment should be preceded by a prophylactic steroid regimen on the day prior to administration and on the day of administration, preferably about one hour prior to infusion. This dosing schedule can yield a dose intensity of IMGN901 of at least about 540 mg/m² over 12 weeks.

Known corticosteroids commonly used, including dexamethasone, beclomethasone, budesonide, flunisolide, fluticasone propionate, hydroctorisone, methylprednisolone, prednisolone, prednisone and trimacinolione acetonide may be used in the present invention. Dexamethasone is the preferred steroid. Known antihistamines including diphenhydramine may be used in combination with the corticosteroids as a prophylactic pre-treatment in the present invention.

The improved methods of treatment and dosage regimens of the invention with the anti-CD56-maytansinoid conjugate provide at least 25% an increase in dose intensity when compared over a 6 week initial treatment duration to conventional methods.

All references, including publications, patent applications, and patents, cited herein are hereby incorporated by reference to the same extent as if each reference were individually and specifically indicated to be incorporated by reference and were set forth in its entirety herein.

The following examples describe the invention in greater detail and are intended to illustrate but not to limit the invention.

### EXAMPLES

The improved IMGN901 methods of treatment examples disclosed below provide at least a 25% increase in dose intensity when compared over an initial 6 week treatment duration.

### Example 1

### Comparison of IMGN901 Treatment with and without Pretreatment with Corticosteroids and a Reduced Initial Infusion Rate

Data in approximately 150 patients enrolled in three Phase I studies evaluating MM, SCLC and MCC confirm the low systemic toxicity of IMGN901. The most noteworthy adverse events (AEs) were Grade 3 and 4 meningitis-like symptoms associated with headaches. After implementation of a slowed infusion rate and routine steroid prophylaxis prior to treatment according to the dosing schedules of the invention, meningitis-like symptoms have not been reported subsequently and Grade 3 and 4 headache have not been reported at the maximum tolerated dose (MTD) for any study. There were no clinically significant changes in hematological parameters and, in particular, no evidence of myelosuppression. Most AEs experienced to date are consistent with those expected in this patient group of heavily pretreated cancer patients.

In the first study, IMGN901 was initially given to patients by IV infusions without prophylactic measures. Two patients received the drug at 75 mg/m² and one patient at 67.5 mg/m². Each of the three patients appeared to have aseptic/chemical meningitis on Cycle 1 Day 1. Severe headaches occurred in all three patients approximately 8-12 hours after receiving the first infusion of IMGN901. Each of the patients responded well to treatment including Tylenol and Zofran for their meningitis and their symptoms rapidly improved and resolved over 2-5 days.

Subsequently, three additional patients received IMGN901 at a dose of 60 mg/m² and developed grade 3 or 4 headaches that were reported as SAEs after receiving the first infusion on Cycle 1 Day 1. Patients were admitted to the hospital and received treatment for the headaches. All symptoms resolved over several days.

The protocol for this Study was subsequently amended to recommend prophylactic measures according to the invention. The prophylactici measures included dexamethasone on the day prior to and on the day of IMGN901 administration. There were no incidences of severe headache/aseptic meningitis in the further 13 patients treated at the dose of 60 mg/m² with the prophylactic measures under the protocol amendment.

In a second Clinical Study, one patient received the first of three planned daily doses of IMGN901 at 75 mg/m². No prophylactic measures were administered. The patient developed a headache the same day, which worsened despite treatment with paracetamol (acetaminophen), followed by an episode of vomiting. Further treatment with codeine and anti-emetics was given. All symptoms resolved within six days. Given that this patient developed severe headache after IMGN901 was infused over 40 minutes, an additional six patients were treated with IMGN901 at a slower infusion rate of 1 mg/min. No IMGN901-related grade 3 or 4 headaches or other toxicities at the 75 mg/m² dose were observed, although two out of the six patients then treated at this dose with the slower infusion rate did develop grade 2 headaches.

### Example 2

### Method of IMGN901 Treatment on a Day 1, 8, Every 3 Weeks Schedule in Patients with Heavily Pre-Treated CD56-Positive Multiple Myeloma

IMGN901 was administered intravenously as single-agent therapy on days 1 and 8 every 21 days at a dose of at least 90 mg/m², yielding a dose intensity of at least 360 mg/m² over 6 weeks. Three patients each were administered dose levels of 40 mg/m², 60 mg/m², 75 mg/m² and 90 mg/m², while eight patients were administered a dose level of 112 mg/m² and six patients were administered a dose level of 140 mg/m². IMGN901 was infused initially at the rate of 1 mg/min. If tolerated, the initial infusion rate of 1 mg/min was increased up to 3 mg/min. Treatment with IMGN901 was preceded by a prophylactic regimen of corticosteroids. On the day prior to administration of IMGN901, patients received dexamethasone 8 mg (or similar steroid equivalent) by mouth BID. On the day of IMGN901 administration, and approximately one hour prior to the infusion, patients received dexamethasone 10 mg IV (or similar steroid equivalent).

An investigator-reported partial response was observed in a patient treated at 140 mg/m² and the patient has remained on treatment for over a year. Three minor responses were reported in one patient each at doses of 60, 90 and 112 mg/m², with two of these sustained for 45 weeks or longer. Eleven patients had stable disease, with four of these patients having remained on treatment for 24 weeks or longer. Ten patients had IMGN901 treatment duration in excess of some regimens used earlier in the course of their disease. Eight of these ten patients had IMGN901 treatment duration longer than the most recent regimen used to treat their disease (total treatment duration with IMGN901=281 weeks for these 8 patients versus total treatment duration on most previous myeloma regimen=69 weeks). Mild to moderate headache, fatigue and neuropathy, and some mild, transient lab abnormalities were the most commonly reported adverse events related to IMGN901.

### Example 3

### Method of IMGN901 Treatment on a Day 1, 2, 3 Every 3 Weeks Schedule in Patients with CD56-Positive Solid Tumors

IMGN901 was administered intravenously as single-agent therapy daily for 3 days every 3 weeks. IMGN901 was administered at a dose of at least 60 mg/m², yielding a dose intensity of at least 360 mg/m² over 6 weeks. Patients were dosed as follows:

| | Dose (mg/m²) | | | | | | | | | |
|---|---|---|---|---|---|---|---|---|---|---|
| | 4 | 8 | 16 | 24 | 36 | 48 | 60 | 75 | 94 | Total |
| Patients (n) | 4 | 4 | 6 | 4 | 4 | 7 | 11 | 22 | 2 | 64 |

IMGN901 is infused initially at the rate of 1 mg/min. If tolerated, the initial infusion rate of 1 mg/min was increased up to 3 mg/min. Treatment with IMGN901 was preceded by a prophylactic corticosteroid regimen. On the day prior to administration of IMGN901, patients received dexamethasone 8 mg (or similar steroid equivalent) by mouth BID. On the day of IMGN901 administration, and approximately one hour prior to the infusion, patients received dexamethasone 10 mg IV (or similar steroid equivalent).

One Merkel Cell Carcinoma (“MCC”) patient treated at 36 mg/m² attained a complete response and has been disease free in excess of five years without any subsequent treatment. One MCC patient treated at 60 mg/m² had a partial response that evolved into a complete response based on clinical examination and has been disease-free for seventeen months. One small cell lung cancer (“SCLC”) patient treated at 75 mg/m² had an unconfirmed partial response (patient had refractory disease). In total, thirteen patients experienced some reduction in tumor volume that ranged from 1.3 to 100 percent. In addition, to the objective responses, 27 patients had protocol-defined stable disease based on clinical assessment and tumor measurements. These included: (i) three MCC patients (two who received four cycles of treatment and one who has received seven cycles of treatment) and (ii) three SCLC patients with stable disease lasting at least 90 days. Six patients received IMGN901 longer than their most recent prior therapy.

### Example 4

### Method of IMGN901 Treatment on a Day 1, 8, 15 Every 4 Weeks Schedule in. Combination with Another Anti-Cancer Agent

IMGN901 will be administered intravenously on days 1, 8, 15 every 4 weeks in combination with lenalidomide and dexamethasone. IMGN901 will be administered at a dose of at least 45 mg/m² over 12 weeks. IMGN901 will be infused initially at the rate of 1 mg/min. If tolerated, the initial infusion rate of 1 mg/min or lower will be increased up to 3 mg/min. The initial infusion rate will be increased incrementally, preferably in increments of 0.5 mg/min. Doses of IMGN901 that can be administered in accordance with the invention are 45 mg/m², 60 mg/m², 75 mg/m², 90 mg/m² and 112 mg/m². It is expected that the dose intensity of IMGN901 over 12 weeks will be at least 405 mg/m², 540 mg/m², 675 mg/m², 810 mg/m² or 1008 mg/m², depending on the specific dosing schedule employed. Lenalidomide will be administered at a dose of 25 mg once daily on days 1 to 21, every four weeks and dexamethasone may be administered at a dose of 40 mg once daily on days 1, 8, 15 and 22, every 4 weeks. Both Lenalidomide and dexamethasone will be administered about 30 minutes prior to infusion with IMGN901. Treatment should be preceded by a prophylactic steroid regimen. On the day prior to administration of IMGN901, the patients should receive dexamethasone 8 mg (or similar steroid equivalent) by mouth BID and, on the day of administration, approximately one hour prior to the infusion, patients should receive dexamethasone 10 mg IV (or similar steroid equivalent). If patients receive a dose of dexamethasone at 40 mg, then the dose of dexamethasone at 10 mg IV one hour prior to the infusion will be omitted.

The study will aim to identify a dosing regimen that will improve the efficacy of a current treatment regimen in multiple myeloma. Efficacy will be assessed based on tumor response through measuring parameters such as decrease in myleoma proteins in the blood and urine, improvement in progression-free survival, time to progression and overall survival.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method for treating cancer, comprising pre-treating a subject in need of treatment with prophylactic corticosteroids and subsequently administering an anti-CD56-maytansinoid conjugate by infusion at an initial infusion rate of 1 mg/min or lower on a schedule selected from the group consisting of: (1) an amount of at least about 90 mg/m² on day 1 and day 8, every three weeks; and (2) at least an amount of about 30 mg/m² on day 1, day 2 and day 3, every three weeks.

2. The method of claim 1, wherein said method of treating cancer further comprises treating mammalian tumors.

3. The method according to claim 2, wherein the anti-CD56-maytansinoid conjugate is IMGN901.

4. The method according to claim 2, wherein the anti-CD56-maytansinoid conjugate is administered at a dose selected from the group consisting of:
(a) at least about 90 mg/m² on day 1 and day 8 every three weeks, and
(b) at least about 112 mg/m² on day 1 and day 8 every three weeks.

5. The method according to claim 2, wherein the anti-CD56-maytansinoid conjugate is administered at a dose selected from the group consisting of:
(a) at least about 30 mg/m² on day on day 1, day 2 and day 3, every three weeks,
(b) at least about 36 mg/m² on day on day 1, day 2 and day 3, every three weeks,
(c) at least about 48 mg/m² on day on day 1, day 2 and day 3, every three weeks,
(d) at least about 60 mg/m² on day on day 1, day 2 and day 3, every three weeks, and
(e) at least about 75 mg/m² on day on day 1, day 2 and day 3, every three weeks.

6. The method according to claim 2, wherein the cancer is selected from the group consisting of small cell lung cancer; ovarian cancer; non small cell lung cancer; neuroendocrine tumors selected from the group consisting of Merkel cell carcinoma, large cell neuroendocrine carcinoma of the lung, neuroendocrine tumors of the pancreas and gastro-intestinal tract; breast cancer; typical and atypical carcinoid of the lung; neuroblastoma; sarcomas; osteosarcomas; astrocytomas; Wilms tumor; schwannoma; multiple myeloma; Natural Killer (NK) cell lymphoma; and acute myelocytic leukemia.

7. The method according to claim 2, wherein the corticosteroid is selected from the group consisting of dexamethasone, beclomethasone, budesonide, flunisolide, fluticasone propionate, hydroctorisone, methylprednisolone, prednisolone, prednisone and trimacinolione acetonide.

8. The method according to claim 7, wherein the corticosteroid is dexamethasone.

9. The method according to claim 2, further comprising administration of an anti-cancer agent.

10. The method according to claim 2, wherein the pre-treating further comprises an antihistamine in combination with the corticosteroid.

11. The method according to claim 2, wherein the antihistamine is diphenhydramine.

12. The method according to claim 2, further comprising increasing the infusion rate incrementally up to 3 mg/min if the initial infusion rate is tolerated.

13. The method according to claim 2, further comprising increasing the infusion rate up to 3 mg/min in increments of 0.5 mg/min if the initial fusion rate is tolerated.

14. A dosing regimen for the treatment of cancer, comprising pre-treating a subject in need of treatment with prophylactic corticosteroids and subsequently administering an anti-CD56-maytansinoid conjugate by infusion at an initial infusion rate of 1 mg/min or lower on a schedule selected from the group consisting of: (1) an amount of at least about 90 mg/m² on day 1 and day 8, every three weeks; and (2) an amount of at least about 30 mg/m² on day 1, day 2 and day 3, every three weeks.

15. The dosing regimen of claim 14, wherein said treatment of cancer further comprises the treatment of mammalian tumors.

16. The method according to claim 14, wherein the anti-CD56-maytansinoid conjugate is IMGN901.

17. The method according to claim 14, wherein the anti-CD56-maytansinoid conjugate is administered at a dose selected from the group consisting of:
(a) at least about 90 mg/m² on day 1 and day 8 every three weeks and
(b) at least about 112 mg/m² on day 1 and day 8 every three weeks.

18. The method according to claim 14, wherein the anti-CD56-maytansinoid conjugate is administered at a dose selected from the group consisiting of:
(a) at least about 30 mg/m² on day on day 1, day 2 and day 3, every three weeks;
(b) at least about 36 mg/m² on day on day 1, day 2 and day 3, every three weeks;
(c) at least about 48 mg/m² on day on day 1, day 2 and day 3, every three weeks;
(d) at least about 60 mg/m² on day on day 1, day 2 and day 3, every three weeks; and
(e) at least about 75 mg/m² on day on day 1, day 2 and day 3, every three weeks.

19. The method according to claim 14, wherein the cancer is selected from the group consisting of small cell lung cancer; ovarian cancer; non small cell lung cancer; neuroendocrine tumors selected from the group consisting of Merkel cell carcinoma, large cell neuroendocrine carcinoma of the lung, neuroendocrine tumors of the pancreas and gastro-intestinal tract; breast cancer; typical and atypical carcinoid of the lung; neuroblastoma; sarcomas; osteosarcomas; astrocytomas; Wilms tumor; schwannoma; multiple myeloma; Natural Killer (NK) cell lymphoma; and acute myelocytic leukemia.

20. The method according to claim 14, wherein the corticosteroid is selected from the group consisting of dexamethasone, beclomethasone, budesonide, flunisolide, fluticasone propionate, hydroctorisone, methylprednisolone, prednisolone, prednisone and trimacinolione acetonide.

21. The method according to claim 20, wherein the corticosteroid is dexamethasone.

22. The method according to claim 14, wherein the pre-treating further comprises an antihistamine in combination with the corticosteroid.

23. The method according to claim 22, wherein the antihistamine is diphenhydramine.

24. The method according to claim 14, further comprising increasing the infusion rate incrementally up to 3 mg/min if the initial infusion rate is tolerated.

25. The method according to claim 14, further comprising increasing the infusion rate in increments of 0.5 mg/min up to 3 mg/min if the initial infusion rate is tolerated.

26. A method for treating cancer, comprising pre-treating a subject in need of treatment with prophylactic corticosteroids and subsequently administering an anti-CD56-maytansinoid conjugate by infusion at an initial infusion rate of 1 mg/min or lower in combination with another anticancer treatment on a schedule selected from the group consisting of: (1) an amount of at least about 45 mg/m² on day 1, day 8 and day 15, every four weeks; and (2) at least an amount of about 45 mg/m² on day 1 and day 8, every three weeks.

27. The method of claim 26, wherein said method of treating cancer further comprises treating mammalian tumors.

28. The method according to claim 26, wherein the anti-CD56-maytansinoid conjugate is IMGN901.

29. The method according to claim 26, wherein the anti-CD56-maytansinoid conjugate is administered at a dose selected from the group consisting of:
(a) at least about 45 mg/m² on day 1, day 8, and day 15, every four weeks
(b) at least about 60 mg/m² on day 1, day 8, and day 15, every four weeks;
(c) at least about 45 mg/m² on day 1, day 8, and day 15, every four weeks;
(d) at least about 60 mg/m² on day 1, day 8, and day 15, every four weeks;
(e) at least about 75 mg/m² on day 1, day 8, and day 15, every four weeks;
(f) at least about 90 mg/m² on day 1, day 8, and day 15, every four weeks; and
(g) at least about 112 mg/m² on day 1, day 8, and day 15, every four weeks.

30. The method according to claim 26, wherein the anti-CD56-maytansinoid conjugate is administered at a dose selected from the group consisting of:
(a) at least about 45 mg/m² on day 1 and on day 8, every three weeks;
(b) at least about 60 mg/m² on day 1 and on day 8, every three weeks;
(c) at least about 75 mg/m² on day 1 and on day 8, every three weeks;
(d) at least about 90 mg/m² on day 1 and on day 8, every three weeks; and
(e) at least about 112 mg/m² on day 1 and on day 8, every three weeks.

31. The method according to claim 26, wherein said another anticancer treatment is lenalidomide and dexamethasone.

32. The method according to claim 31, wherein lenalidomide is administered in an amount of about 25 mg daily on days 1 to 21, every four weeks.

33. The method according to claim 31, wherein dexamethasone is administered in an amount of about 40 mg daily on day 1, day 8, day 15 and day 22, every four weeks.

34. The method according to claim 32, wherein dexamethasone is administered in an amount of about 40 mg daily on day 1, day 8, day 15 and day 22, every four weeks.

35. The method according to claim 31, wherein lenalidomide and dexamethasone are administered orally.

36. The method according to claim 26, wherein said another anticancer treatment is etoposide and carboplatin.

37. The method according to claim 36, wherein etoposide is administered in an amount of about 75-120 mg/m² on day 1, day 2 and day 3 and carboplatin is administered in an amount of about 5-6 AUC on day 1 every three weeks.

38. The method according to claim 37, wherein etoposide is administered in an amount of about 100 mg/m².

39. The method according to claim 37, wherein carboplatin is administered in an amount of about 6 AUC.

40. The method according to claim 38, wherein carboplatin is administered in an amount of about 6 AUC.

41. The method according to claim 36, wherein etoposide is administered orally.

42. The method according to claim 36, where etoposide is administered intravenously.

43. The method according to claim 36, wherein carboplatin is administered intravenously.

44. The method according to claim 26, wherein the cancer is selected from the group consisting of small cell lung cancer; ovarian cancer; non small cell lung cancer; neuroendocrine tumors selected from the group consisting of Merkel cell carcinoma, large cell neuroendocrine carcinoma of the lung, neuroendocrine tumors of the pancreas and gastro-intestinal tract; breast cancer; typical and atypical carcinoid of the lung; neuroblastoma; sarcomas; osteosarcomas; astrocytomas; Wilms tumor; schwannoma; multiple myeloma; Natural Killer (NK) cell lymphoma; and acute myelocytic leukemia.

45. The method according to claim 26, wherein the corticosteroid is selected from the group consisting of dexamethasone, beclomethasone, budesonide, flunisolide, fluticasone propionate, hydroctorisone, methylprednisolone, prednisolone, prednisone and trimacinolione acetonide.

46. The method according to claim 26, wherein the corticosteroid is dexamethasone.

47. The method according to claim 26, wherein the pre-treating further comprises an antihistamine in combination with the corticosteroid.

48. The method according to claim 47, wherein the antihistamine is diphenhydramine.

49. The method according to claim 47, further comprising increasing the infusion rate incrementally up to 3 mg/min if the initial infusion rate is tolerated.

50. The method according to claim 47, further comprising increasing the infusion rate up to 3 mg/min in increments of 0.5 mg/min if the initial infusion rate is tolerated.

51. A dosing regimen for the treatment of cancer, comprising pre-treating a subject in need of treatment with prophylactic corticosteroids and subsequently administering an anti-CD56-maytansinoid conjugate by infusion at an initial infusion rate of 1 mg/min or lower in combination with another anticancer treatment on a schedule selected from the group consisting of: (1) an amount of at least about 45 mg/m² on day 1, day 8 and day 15 every four weeks; and (2) an amount of at least about 45 mg/m² on day 1, and day 8, every three weeks.

52. The dosing regimen of claim 51, wherein said treatment of cancer further comprises the treatment of mammalian tumors.

53. The method according to claim 51, wherein the anti-CD56-maytansinoid conjugate is IMGN901.

54. The method according to claim 51, wherein the anti-CD56-maytansinoid conjugate is administered at a dose selected from the group consisting of:
(a) at least about 45 mg/m² on day 1, day 8, and day 15, every four weeks;
(b) at least about 60 mg/m² on day 1, day 8, and day 15, every four weeks;
(c) at least about 75 mg/m² on day 1, day 8, and day 15, every four weeks;
(d) at least about 90 mg/m² on day 1, day 8, and day 15, every four weeks; and
(e) at least about 112 mg/m² on day 1, day 8, and day 15, every four weeks.

55. The method according to claim 51, wherein the anti-CD56-maytansinoid conjugate is administered at a dose selected from the group consisting of:
(a) at least about 45 mg/m² on day 1 and on day 8, every three weeks;
(b) at least about 60 mg/m² on day 1 and on day 8, every three weeks;
(c) at least about 75 mg/m² on day 1 and on day 8, every three weeks;
(d) at least about 90 mg/m² on day 1 and on day 8, every three weeks; and
(e) at least about 112 mg/m² on day 1 and on day 8, every three weeks.

56. The method according to claim 51, wherein said another anticancer treatment is lenalidomide and dexamethasone.

57. The method according to claim 56, wherein lenalidomide is administered in an amount of about 25 mg daily on days 1 to 21, every four weeks.

58. The method according to claim 56, wherein dexamethasone is administered in an amount of about 40 mg daily on day 1, day 8, day 15 and day 22, every four weeks.

59. The method according to claim 57, wherein dexamethasone is administered in an amount of about 40 mg daily on day 1, day 8, day 15 and day 22, every four weeks.

60. The method according to claim 56, wherein lenalidomide and dexamethasone are administered orally.

61. The method according to claim 51, wherein said another anticancer treatment is etoposide and carboplatin.

62. The method according to claim 61, wherein etoposide is administered in an amount of about 75-120 mg/m² on day 1, day 2 and day 3 and carboplatin is administered in an amount of about 5-6 AUC on day 1 every three weeks.

63. The method according to claim 62, wherein etoposide is administered in an amount of about 100 mg/m².

64. The method according to claim 62, wherein carboplatin is administered in an amount of about 6 AUC.

65. The method according to claim 63, wherein carboplatin is administered in an amount of about 6 AUC.

66. The method according to claim 61, wherein etoposide is administered orally.

67. The method according to claim 61, where etoposide is administered intravenously.

68. The method according to claim 61, wherein carboplatin is administered intravenously.

69. The method according to claim 51, wherein the cancer is selected from the group consisting of small cell lung cancer; ovarian cancer; non small cell lung cancer; neuroendocrine tumors selected from the group consisting of Merkel cell carcinoma, large cell neuroendocrine carcinoma of the lung, neuroendocrine tumors of the pancreas and gastro-intestinal tract; breast cancer; typical and atypical carcinoid of the lung; neuroblastoma; sarcomas; osteosarcomas; astrocytomas; Wilms tumor; schwannoma; multiple myeloma; Natural Killer (NK) cell lymphoma; and acute myelocytic leukemia.

70. The method according to claim 51, wherein the corticosteroid is selected from the group consisting of dexamethasone, beclomethasone, budesonide, flunisolide, fluticasone propionate, hydroctorisone, methylprednisolone, prednisolone, prednisone and trimacinolione acetonide.

71. The method according to claim 70, wherein the corticosteroid is dexamethasone.

72. The method according to claim 51, wherein the pre-treating further comprises an antihistamine in combination with the corticosteroid.

73. The method according to claim 72, wherein the antihistamine is diphenhydramine.

74. The method according to claim 51, further comprising increasing the infusion rate incrementally up to 3 mg/min if the initial infusion rate is tolerated.

75. The method according to claim 51, further comprising increasing the infusion rate up to 3 mg/min in increments of 0.5 mg/min if the initial infusion rate is tolerated.
